(19) United States
(12) Patent Application Publication
(10) Pub. No.: US 2026/0357117 A1
(43) Pub. Date: Nov. 12, 2026

(54) PROCESSES FOR SEPARATION OF MIXTURES COMPRISING 1,1,1,3-TETRAFLUOROPROPANE, 3,3,3-TRIFLUOROPROPENE, AND HYDROGEN FLUORIDE AND AZEOTROPES THEREOF

(71) Applicant: THE CHEMOURS COMPANY FC, LLC, WILMINGTON, DE (US)

(72) Inventors: XUEHUI SUN, KENNETT SQUASRE, PA (US); MICHAEL A. BRADLEY, HOCKESSIN, DE (US); KARL ROBERT KRAUSE, KENNETT SQUASRE, PA (US)

(73) Assignee: THE CHEMOURS COMPANY FC, LLC, WILMINGTON, DE (US)

(21) Appl. No.: 19/118,155

(22) PCT Filed: Nov. 14, 2023
(86) PCT No.: PCT/US2023/037237

§ 371 Date: Apr. 3, 2025

Related U.S. Application Data
(60) Provisional application No. 63/426,596, filed on Nov. 18, 2022.

(51) Int. Cl. C09K 5/04

(52) U.S. Cl. CPC C09K 5/044; C09K 2205/22; C09K 2205/32

(57) ABSTRACT

Disclosed herein are processes for separating hydrogen fluoride (HF) from a composition including 1,1,1,3-tetrafluoropropane (HFC-254fb), 3,3,3-trifluoropropene (HFO-1243zf), and a molar excess of HF, including azeotropic distillation. Also disclosed herein are processes for separating HFC-254fb from an HFC-254fb-rich stream including HFC-254fb, HFO-1243zf, and HF, including azeotropic distillation. Also disclosed herein are processes for separating HFC-254fb from a process stream including HFC-254fb, HFO-1243zf, and a molar excess of HF including azeotropic distillation. Also disclosed herein are azeotrope or near-azeotrope compositions including HFC-254fb/HF, HFO-1243zf/HF, mixtures of HFC-254fb/HF, HFO-1243zf/HF, and HF.

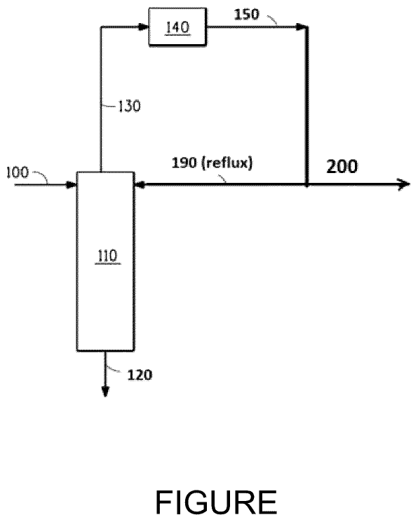

### CROSS REFERENCE TO RELATED APPLICATIONS

This application claims priority to U.S. Provisional Application No. 63/426,596, filed on Nov. 18, 2022, the disclosure of which is herein incorporated by reference in its entirety.

### FIELD

### Field of the Disclosure

This disclosure relates to processes for separating mixtures of hydrogen fluoride (HF) and fluoroolefins. More specifically, this disclosure relates to processes for separating mixtures including 1,1,1,3-tetrafluoropropane (HFC-254fb; CF₃CH₂CH₂F), 3,3,3-trifluoropropene (HFO-1243zf; CF₃CH═CH₂) and HF. This disclosure also relates to azeotrope or near-azeotrope mixtures for separation.

### Background

The refrigeration industry continues to address factors which affect the environment, by using refrigerants which have minimal or no ozone depleting potential (ODP), and more recently, developing materials and processes which have low global warming potential (GWP). To alleviate these environmental concerns, there continues to be a need for heat transfer compositions that possess low global warming potentials (“GWP”) and maintain or improve performance. While certain hydrofluoroolefins (HFOs) have low GWPs, there remains a need for manufacturing processes that provide fluoroolefins that have lower global warming potential.

The manufacture of fluoroolefins is typically a multi-step process that may produce intermediate mixtures of hydrofluorocarbons (HFCs), hydrochlorofluorocarbons (HCFCs), hydrochlorofluoroolefins (HCFOs), and/or hydrofluoroolefins (HFOs), and hydrogen fluoride (HF). The separation of such mixtures is not always easily accomplished. Existing methods of distillation and decantation are very often ineffective for separation of these compounds. Aqueous scrubbing may be effective but requires the use of large amounts of scrubbing solutions and produces excessive waste as well as wet product that must then be dried.

Therefore, there is a need for new methods of separating HF and fluoroolefins, hydrofluoroolefins, hydrofluoroolefins, chlorofluoroolefins, hydrochlorofluoroolefins, hydrochlorofluorocarbons, and/or hydrofluorocarbons.

### SUMMARY

The instant invention solves problems associated with conventional processes and provides compositions and methods for separating HF from fluoroolefin containing compositions produced from processes including, but not limited to, the manufacture of 3,3,3-trifluoropropene (HFO-1243zf, CF₃CH═CH₂), which may be a part of a general process to manufacture 2,3,3,3-tetrafluoropropene (HFO-1234yf; CF₃CF═CH₂). Producing 3,3,3-trifluoropropene can involve the fluorination of 1,1,1,3-tetrachloropropane (HCC-250fb; CCl₃CH₂CH₂Cl), along with formation of the by-product 1,1,1,3-tetrafluoropropane (HFC-254fb).

In one embodiment of the invention disclosed herein, a process stream containing 1,1,1,3-tetrafluoropropane (HFC-254fb) and 3,3,3-trifluoropropene (HFO-1243zf) includes sufficient HF to also form an azeotrope or near-azeotrope composition containing 1,1,1,3-tetrafluoropropane (HFC-254fb).

In one embodiment, the present disclosure provides a process for separating hydrogen fluoride (HF) from a process stream comprising 1,1,1,3-tetrafluoropropane (HFC-254fb), 3,3,3-trifluoropropene (HFO-1243zf), and a molar excess of HF, i.e., a sufficient amount of HF to surprisingly to form azeotrope or near-azeotrope compositions containing at least 1,1,1,3-tetrafluoropropane (HFC-254fb).

The processes disclosed herein comprise distilling a process stream in a first distillation column under the boiling conditions of azeotropes that form in mixtures between HFC-254fb, HFO-1243zf and excess HF, e.g., between HFC-254fb and HF, as well as between HFO-1243zf and HF. The process also comprises forming a first distillate stream comprising a composition of HFC-254fb, HFO-1243zf, and a sufficient amount of HF to form azeotrope or near azeotrope compositions between HFC-254fb and HF, as well as between HFO-1243zf and HF, and a first (bottoms) stream of HF essentially free of HFC-254fb and HFO-1243zf from the first distillation column.

In another embodiment, the present disclosure provides a process for separating 1,1,1,3-tetrafluoropropane (HFC-254fb) from a stream comprising HFC-254fb, 3,3,3-trifluoropropene (HFO-1243zf), and a molar excess of HF by using the HFC-254fb/HF azeotrope or near-azeotrope. One or more additional compounds may be present in the stream including, but not limited to, propane, trifluoroethane, 1,1,1,2-tetrafluoroethane (HFC-134a; CF₃CH₂F), 1,1,3,3,3-pentafluoropropene (HFO-1225ZC), difluoroethane, 1,1,1-trifluoropropane (HFC-143a; CH₃CH₂CF₃), monofluoroethane (HFC-161), 1,1,1,2-tetrafluoropropane (HFC-254eb), 2-chloro-3,3,3-trifluoropropene (HFO-1233xf), 1-chloro-3,3,3-trifluoropropene (HFO-1233zd), 3-chloro-3,3-difluoropropene (HCFO-1242zf), 1-chloro-1-fluoroethane (HCFC-151a), 1-chloro-2-fluoroethane (HCFC-151), 1-chloro-3,3,3-trifluoropropane (HCFC-253fb), PCE, HCE/HCB, chloroethylene, 1,1,3-trichloro-1-propene (HCC-1240za), other tetra- or penta-fluoropropenes, and combinations thereof.

One process embodiment disclosed herein comprises subjecting the HFC-254fb containing stream to distillation in a first distillation column under the boiling conditions to provide sufficient HF to form HFC-254fb and HFO-1243zf azeotropes or near azeotropes with HF. The process also comprises forming a first (overhead) distillate stream comprising a mixture of the HFC-254fb, HFO-1243zf and sufficient HF such that 254fb/HF and HFO-1243zf/HF azeotropes and near azeotropes form, and forming a first bottom stream of HF essentially free of HFO-1243zf and HFC-254fb. The first distillate stream is separated into an HF-enriched stream and an HFC-254fb-enriched stream which is further processed in a second distillation column to form a second distillate stream and a second bottoms stream which is subsequently distilled in a third distillation column to form an HFC-254fb stream essentially free of HFO-1243zf and HF.

The process further comprises forming a second distillate stream of HFC-254fb, HFO-1243zf, and HF to form an azeotrope or near-azeotrope composition e.g., HFC-254fb/HF and HFO-1243zf/HF, and a second bottoms stream of HFC-254fb and HFO-1243zf essentially free of HF from the second distillation column.

In certain embodiments the second bottoms stream can be distilled by standard distillation techniques in a third distillation column (not shown) to form an HFC-254fb stream essentially free of HFO-1243zf.

In another embodiment, the present disclosure provides an azeotrope or near-azeotrope composition comprising 1,1,1,3-tetrafluoropropane (HFC-254fb), 3,3,3-trifluoropropene (HFO-1243zf), and hydrogen fluoride (HF), e.g., a mixture of HFC-254fb/HF and HFO-1243zf/HF azeotropes or near-azeotropes.

In another embodiment, the present invention provides an azeotrope or near-azeotrope composition comprising 1,1,1,3-tetrafluoropropane (HFC-254fb) and hydrogen fluoride (HF).

In one particular aspect of this embodiment, the present invention provides a composition which comprises about 18.7 mol % HFC-254fb and about 81.3 mol % HF at a temperature of about 30 °C and has a vapor pressure of about 22.5 psia (169 kPa).

In another particular aspect of this embodiment, the present invention provides a composition comprising: i) an azeotrope or near-azeotrope composition comprising 1,1,1,3-tetrafluoropropane (HFC-254fb) and HF, and one or more of ii) HFO-1242zf; and iii) HFO-1243zf.

In one aspect of the invention, the process stream includes a sufficient amount of HF, meaning a molar excess of HF relative to the organic content of the product stream, such that enough HF is present to remove all the organics as azeotrope or near-azeotrope mixtures in the first distillate.

In one embodiment of the invention, the process stream comprises compositions which include a mixture of two azeotropes or near-azeotropes HFC-254fb/HF and HFO-1243zf/HF. In this embodiment, HF is present in an amount sufficient to form both azeotropes or near-azeotropes, and the distillate comprises both azeotropes or near-azeotropes, and the bottoms stream comprises HF essentially free of HFC-254fb and HFO-1243zf. Since HFC-254fb has a higher boiling point than HF, an HFC-254fb/HF azeotrope or near azeotrope composition can be used to separate the HFC-254fb.

Other features and benefits of any one or more of the embodiments described herein will be apparent from the following detailed description, and from the claims.

The various embodiments of the invention can be used alone or in combinations with each other. The foregoing general description and the following detailed description are exemplary and explanatory only and are not restrictive of the invention, as defined in the appended claims.

### BRIEF DESCRIPTION OF THE DRAWINGS

An embodiment is illustrated in the accompanying FIGURE to improve understanding of concepts as presented herein.

The FIGURE illustrates an embodiment of an azeotrope distillation for the separation of HFC-254fb from a mixture containing HFC-254fb, HFO-1243zf, and HF.

Skilled artisans appreciate that objects in the FIGURE are illustrated for simplicity and clarity and have not necessarily been drawn to scale. For example, the dimensions of some of the objects in the FIGURE may be exaggerated relative to other objects to help to improve understanding of embodiments.

### DETAILED DESCRIPTION OF THE INVENTION

Described herein are processes for separating HFC-254fb, HFO-1243zf, and HF from a composition comprising HFC-254fb, HFO-1243zf, and HF. The processes take advantage of the unexpected formation of the azeotrope or near-azeotrope compositions of HFC-254fb and HF. Since HFC-254fb surprisingly forms low boiling azeotrope or near-azeotrope mixtures with HF, these mixtures can be used to produce HF essentially free of HFC-254fb and HFO-1243zf in a single distillation, even though HFC-254fb has a higher boiling point than HF and HFO-1243zf has a lower boiling point than HF.

The processes disclosed herein generally comprise subjecting a composition comprising HFC-254fb, HFO-1243zf, and HF to a distillation step; forming a distillate composition comprising HF, HFC-254fb and HFO-1243zf which forms an azeotrope or near-azeotrope composition comprising HFC-254fb and HF, and which can also include a mixture of azeotrope or near-azeotrope compositions, e.g., HFC-254fb/HF and HFO-1243zf/HF; and forming a bottoms composition comprising HF essentially free of either HFC-254fb or HFO-1243zf.

Described herein are azeotrope and azeotrope-like (used interchangeably herein with “near-azeotrope”) compositions comprising HFC-254fb and HF, alone or in admixture with an HFO-1243zf/HF azeotrope.

Described herein are azeotrope and azeotrope-like compositions comprising HFC-254fb and HF, and more particularly an HFC-254fb/HF azeotrope or near-azeotrope.

Described herein are compositions comprising an HFC-254fb/HF azeotrope or near-azeotrope alone or in admixture with an HFO-1243zf/HF azeotrope or near-azeotrope.

Also described herein are azeotrope and azeotrope-like compositions suitable for separating HFC-254fb.

Also described herein are azeotrope and azeotrope-like compositions to be separated.

In some embodiments, the composition comprising HFC-254fb, HFO-1243zf, and HF is a process stream.

In some embodiments, the composition to be separated contains additional HFC-254fb or HF, beyond the amount necessary to form the azeotrope or azeotrope-like composition including HFC-254fb/HF and optionally HFO-1243zf/HF.

Before addressing details of embodiments described below, some terms are defined or clarified.

By “azeotrope” composition is meant a constant boiling liquid admixture of two or more substances that behaves as a single substance. One way to characterize an azeotrope composition is that the vapor produced by partial evaporation or distillation of the liquid has the same composition as the liquid from which it was evaporated or distilled, that is, the admixture distills/refluxes without compositional change. Constant boiling compositions are characterized as azeotropic because they exhibit either a maximum or minimum boiling point, as compared with that of the non-azeotropic mixtures of the same components. Azeotrope compositions are also characterized by a minimum or a maximum in the vapor pressure of the mixture relative to the vapor pressure of the neat components at a constant temperature. For a binary system such as HF and HFC-254fb, the presence of a maximum or minimum pressure as the composition is varied in a PTx measurement is sufficient to indicate that an azeotrope exists.

By “azeotropic” composition is meant a constant boiling liquid admixture of two or more substances that behaves as a single substance. In general, azeotropy is the phenomenon where a composition comprising two or more molecular species such that the relative volatility between any binary pair of components is unity. That is, the composition of a boiling liquid mixture exhibiting azeotropy is identical to the vapor phase that is produced.

$${α}_{ij} = \frac{{K}_{i}}{{K}_{j}} = \frac{{y}_{i} / {x}_{i}}{{y}_{j} / {x}_{j}} = 1 ∀ \{ i , j \} ,$$

where: αᵢⱼ is the relative volatility between component i and component j, Kᵢ is the K factor for component i, Kⱼ is the K factor for component j, yᵢ and xᵢ are the vapor and liquid molar fractions of component i, yⱼ and xⱼ are the vapor and liquid molar fractions of component j.

Additionally, the temperature of a boiling mixture exhibiting azeotropy is constant at a constant pressure. A system is azeotropic when it can be distilled (or condensed) without change of composition. The notion of a system that is “azeotrope-like” or exhibiting “near azeotropy” is commonly known as a system close enough to azeotropy such that the compositions of the liquid and vapor phases in phase equilibrium are of very similar compositions such that during a boiling process the boiling temperature only rises to a small degree. Therefore, all relative volatilities for all i-j binary pairs of the system in the above equation will be very close to unity. Thus, one way to characterize an azeotropic composition is that the vapor produced by partial evaporation or distillation of the liquid has the same composition as the liquid from which it was evaporated or distilled, that is, the admixture distills/refluxes without compositional change. Constant boiling compositions are characterized as azeotropic because they exhibit either a maximum or minimum boiling point, as compared with that of the non-azeotropic mixtures of the same components. Azeotropic compositions are also characterized by a minimum or a maximum in the vapor pressure of the mixture relative to the vapor pressure of the neat components at a constant temperature.

By “azeotrope-like” composition (sometimes referred to as “near-azeotrope”) is meant a constant boiling, or substantially constant boiling, liquid admixture of two or more substances that behaves as a single substance. One way to characterize an azeotrope-like or near-azeotrope composition is that the vapor produced by partial evaporation or distillation of the liquid has substantially the same composition as the liquid from which it was evaporated or distilled, that is, the admixture distills/refluxes without substantial composition change. Another way to characterize an azeotrope-like or near-azeotrope composition is that the bubble point vapor pressure and the dew point vapor pressure of the composition at a particular temperature are substantially the same, for example within 3 percent, as discussed below. Preferably, the terms “azeotrope-like composition” and “near-azeotrope composition” shall be understood to mean a composition wherein the difference between the bubble point pressure (“BP”) and dew point pressure (“DP”) of the composition at a particular temperature is less than or equal to 5 percent based upon the bubble point pressure, i.e., [(BP-DP)/BP]×100≤5, and more preferably a composition wherein the difference between the bubble point pressure (“BP”) and dew point pressure (“DP”) of the composition at a particular temperature is less than or equal to 3 percent based upon the bubble point pressure, i.e., [(BP-DP)/BP]×100 is 3. An azeotrope-like or near-azeotrope composition can also be characterized by the area that is adjacent to the maximum or minimum vapor pressure in a plot of composition vapor pressure at a given temperature as a function of mole fraction of components in the composition.

For compositions that are azeotropic, there is usually some range of compositions around the azeotrope point that, for a maximum boiling azeotrope, have boiling points at a particular pressure higher than the pure components of the composition at that pressure and have vapor pressures at a particular temperature lower than the pure components of the composition at that temperature, and that, for a minimum boiling azeotrope, have boiling points at a particular pressure lower than the pure components of the composition at that pressure and have vapor pressures at a particular temperature higher than the pure components of the composition at that temperature. Boiling temperatures and vapor pressures above or below that of the pure components are caused by unexpected intermolecular forces between and among the molecules of the compositions, which can be a combination of repulsive and attractive forces such as van der Waals forces and hydrogen bonding.

It is recognized in the art that both the boiling point and the amount of each component of an azeotrope composition can change when the azeotrope liquid composition is subjected to boiling at different pressures. Thus, an azeotrope composition may be defined in terms of the unique relationship that exists among components or in terms of the exact amounts of each component of the composition characterized by a fixed boiling point at a specific pressure. An azeotrope or azeotrope-like composition of two or more compounds can be characterized by defining compositions characterized by a boiling point at a given pressure, thus providing identifying characteristics without unduly limiting the scope of the invention by a specific numerical composition, which is limited by and is only as accurate as the analytical equipment available.

It is recognized in this field that when the relative volatility of a system approaches 1.0, the system is defined as forming an azeotrope-like or near-azeotrope composition. Relative volatility is the ratio of the volatility of a first component to the volatility of a second component. The ratio of the mole fraction of a component in vapor to that in liquid is the volatility of the components. To determine the relative volatility of any two compounds, a method known as the PTx method can be used. In this procedure, the total absolute pressure in a cell of known volume is measured at a constant temperature for various compositions of the two compounds. Use of the PTx Method is described in detail in “Phase Equilibrium in Process Design”, Wiley-Interscience Publisher, 1970, written by Harold R. Null, on pages 124 to 126; hereby incorporated by reference. These measurements can be converted into equilibrium vapor and liquid compositions in the PTx cell by using an activity coefficient equation model, such as the Non-Random, Two-Liquid (NRTL) equation, to represent liquid phase nonidealities. Use of an activity coefficient equation, such as the NRTL equation is described in detail in “The Properties of Gases and Liquids,” 4th edition, published by McGraw Hill, written by Reid, Prausnitz and Poling, on pages 241 to 387, and in “Phase Equilibria in Chemical Engineering,” published by Butterworth Publishers, 1985, written by Stanley M. Walas, pages 165 to 244. Both aforementioned references are hereby incorporated by reference.

Without wishing to be bound by any theory or explanation, it is believed that the NRTL equation, together with the PTx cell data, can sufficiently predict the relative volatilities of the HFO-1243zf-containing and HFC-254fb-containing compositions of the present disclosure and can therefore predict the behavior of these mixtures in multi-stage separation equipment such as distillation columns. In addition, the presence of a maximum or minimum pressure in a binary system (such as HF and HFC-254fb) as the composition is varied in a PTx measurement is sufficient to indicate that an azeotrope exists. See for example, U.S. Pat. No. 8,486,293 and International Publication No. WO2009105517, the disclosure of each being incorporated herein by reference which rely on PTx data to predict mixtures, e.g., 254eb and 1243zf with HF.

The conditions and compositions for HF/HFC-254fb azeotropes, according to the present invention, determined from PTx data is provided in Table A.

TABLE A
HF/HFC-254fb

| Temperature, | Pressure, | Mole % | Mole % |
|---|---|---|---|
| °C | psia | HF | HFC-254fb |
| −20.0 | 3.0 | 94.0 | 6.0 |
| −12.8 | 4.2 | 92.1 | 7.9 |
| 11.7 | 12.3 | 85.6 | 14.4 |
| 19.4 | 16.6 | 83.7 | 16.3 |
| 30.2 | 24.5 | 81.3 | 18.7 |
| 40.8 | 35.1 | 79.3 | 20.7 |
| 51.2 | 48.7 | 77.5 | 22.5 |
| 61.4 | 65.7 | 76.1 | 23.9 |
| 71.6 | 86.9 | 74.9 | 25.1 |
| 80.0 | 108.0 | 74.4 | 25.6 |

Based upon these findings, the present invention provides an azeotropic or near-azeotropic composition comprising, consisting of or consisting essentially of about 6.0 to about 25.6 mole percent HFC-254fb and from about 94.0 to about 74.4 mole percent HF.

In some embodiments, the present invention provides an azeotropic or near-azeotropic composition comprising, consisting of or consisting essentially of about 6.0 to about 25.6 mole percent HFC-254fb and from about 94.0 to about 74.4 mole percent HF, and having a boiling point of from about 80 °C at about 108 psia (744.6 kPa) to about −20 °C at about 3.0 psia (20.7 kPa).

As used herein, the term “azeotrope” is meant to refer to azeotrope compositions, azeotrope-like compositions, azeotrope composition and/or near-azeotrope composition.

The process equipment for all the processes disclosed herein and the associated feed lines, effluent lines and associated units may be constructed of materials resistant to hydrogen fluoride. Typical materials of construction, well-known to the art, include carbon steel, stainless steels, in particular of the austenitic type, and the well-known high nickel alloys such as Monel® nickel-copper alloys, Hastelloy® nickel-based alloys and Inconel® nickel-chromium alloys.

By azeotropic distillation is meant a process in which a distillation column is operated under conditions to cause one or more azeotrope or azeotrope-like compositions to form, and thereby facilitates the separation of the components of the mixture. Azeotrope distillations may occur where only the components of the mixture to be separated are distilled, or where an entrainer is added that forms an azeotrope with one or more of the components of the initial mixture. Entrainers that act in this manner, that is to say, that form an azeotrope with one of more of the components of the mixture to be separated thus facilitating the separation of those components by distillation, are more commonly called azeotroping agents or azeotrope entrainers.

In conventional or azeotrope distillations, the overhead or distillate stream exiting the column may be condensed using conventional reflux condensers. At least a portion of this condensed stream can be returned to the top of the column as reflux, and the remainder recovered as product or for optional processing. The ratio of the condensed material which is returned to the top of the column as reflux to the material removed as distillate is commonly referred to as the reflux ratio. The compounds and entrainer exiting the column as distillate or distillation bottoms stream can then be passed to a stripper or second distillation column for separation by using conventional distillation, or may be separated by other methods, such as decantation. If desired, the entrainer may then be recycled back to the first distillation column for reuse. In some embodiments, the composition and the separation process are free of or essentially free of an added entrainer.

The specific conditions which can be used for practicing the invention depend upon a number of parameters, such as the diameter of the distillation column, feed points, number of separation stages in the column, among others. In some embodiments, the operating pressure of the distillation system may range from about 5 to about 500 psia (34 to 3450 kPa), in another embodiment, about 20 to about 400 psia (140 to 2760 kPa). Normally, increasing the reflux ratio results in increased distillate stream purity, but generally the reflux ratio ranges between about 1/1 to about 200/1. The temperature of the condenser, which is located adjacent to the top of the column, is normally sufficient to substantially fully condense the distillate that is exiting from the top of the column or is the temperature required to achieve the desired reflux ratio by partial condensation.

As used herein, by “essentially free of” is meant that a composition contains less than about 100 ppm (mole basis), less than about 10 ppm or less than about 1 ppm of the specified component. If a composition is essentially free of more than one component, then the total concentration of those components is less than about 100 ppm, less than about 10 ppm, or less than about 1 ppm.

Hydrogen fluoride (HF, anhydrous) is a commercially available chemical or can be produced by methods known in the art.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition, method that includes materials, steps, features, components, or elements, in addition to those literally disclosed provided that these additional included materials, steps, features, components, or elements do not materially affect the basic and novel characteristic(s) of the claimed invention, especially the mode of action to achieve the desired result of any of the processes of the present invention. The term ‘consisting essentially of’ occupies a middle ground between “comprising” and ‘consisting of’.

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present disclosure, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

“Molar excess of HF” means an amount of HF in excess of that necessary to form an azeotrope or a near azeotrope mixture with the organic (HFC or HFO) present in a composition. The molar amount of HF will vary as a function of the organic present in the composition to be separated as well as separation conditions. The molar excess of HF for a given composition is an amount of HF greater than the amount of HF which is required to form an azeotrope (or near azeotrope mixture) for each organic compound in the composition. Reference is made to the following list of the amounts of HF required to form an azeotrope with each of HFO-1243zf, HFC-254eb, and HFC-254fb at 100 psig, which may change with pressure.

TABLE 1

| | Azeotrope | | |
|---|---|---|---|
| Organic Azeotrope | Mol % HF | Azeotrope B.P. | Organic B.P. |
| Component | @100 psig | @ 100 psig | @100 psig |
| 1243zf (CF3CH═CH2) | 27.7 | 32.6 °C | 36.0 °C |
| 254eb (CF3CHFCH3) | 41.6 | 56.3 °C | 64.5 °C |
| 254fb (CF3CH2CH2F)** | 74.2 | 82.4 °C | 96.6 °C |

As illustrated above, the amount of HF needed to form an azeotrope composition with an organic, e.g., HFO-1243zf, HFC-254eb, or HFC-254fb, increases from HFO-1243zf to HFC-254fb. In other words, more than twice as much HF is needed to form an HFC-254fb/HF azeotrope or near-azeotrope composition as compared to an azeotrope or near-azeotrope composition of HFO-1243zf/HF. As shown by the above Table 1, the mol % content of HF in the azeotrope increases from about 28% at 100 psig for HFO-1243zf to about 74% for HFC-254fb, according to the relation HFO-1243zf<HFC-254eb<HFC-254fb which follows the expected order from lowest to highest boiling point of the organic component.

Thus, in some embodiments, in order to provide a bottoms stream where HF is free of or essentially free of both HFO-1243zf and HFC-254fb, the amount of HF in the feed stream is at least equal to the amount of HF needed for both an HFO-1243zf/HF and an HFC-254fb/HF azeotrope or near-azeotrope. An HFO-1243zf/HF azeotrope can form in the absence of an HFC-254fb/HF azeotrope when the amount of HF in the feed stream is less than the amount of HF in the HFO-1243zf/HF azeotrope. However, under these conditions, the bottoms stream from the distillation column will contain HFC-254fb. If, an HFC-254fb/HF azeotrope is present, then an HF feed content that is more than the amount of HF in the HFO-1243zf/HF azeotrope but less than the amount of HF in the HFC-254fb/HF azeotrope will still result in a bottoms stream comprising HFC-254fb. Only when the amount of HF in the feed stream is greater than the total amount of HF in both the HFO-1243zf/HF and an HFC-254fb/HF azeotropes or near-azeotropes can the bottoms stream containing HF be free of or essentially free of HFO-1243zf and HFC-254fb. Therefore, a process stream at 100 psig comprising equimolar amounts of the three organic azeotropes, e.g., 1 mole of each, would require at least 3.97 moles of HF be available for the azeotropes to form. A change in pressure will change the amount of HF that will be necessary for the three azeotropes.

In some embodiments, the separation process is part of a general process to manufacture 3,3,3-trifluoropropene (HFO-1243zf, CF3CH═CH2). In some embodiments, the manufacture of HFO-1243zf is part of a general process to manufacture of 2,3,3,3-tetrafluoropropene (HFO-1234yf; CF3CF═CH2). In some embodiments, the separation process separates a process stream that is a reaction product stream of a reaction process to produce HFO-1243zf. In some embodiments, the reaction process produces HFO-1243zf by fluorination of 1,1,1,3-tetrachloropropane (HCC-250fb; CCl3CH2CH2Cl).

HFO-1243zf may be made by fluorination of HCC-250fb with HF over a fluorination catalyst such as chromium/alumina fluoride or chromium oxide catalysts. HCC-250fb may be made by processes known in the art such as described in U.S. Pat. Nos. 4,605,802 and 5,705,779, which are hereby incorporated by reference in their entireties, by an addition reaction of carbon tetrachloride and ethylene.

The reaction product stream by fluorination of HCC-250fb with HF may include HFC-254fb and other intermediate products in addition to excess HF and the HFO-1243zf product. Other intermediate products may include one or more of 3-chloro-3,3,-difluoropropene (HCFO-1242zf; CF2Cl—CH═CH2), dichlorodifluoromethane (CFC-12; CCl2F2), chlorotrifluoromethane (CFC-13; CClF3), 1,1,1-trifluoroethane (HFC-143a; CF3-CH3), 1,1,1,2,2-pentachloro-2-fluoroethane (HCFC-111; CCl₃CCl2F), 1,2-dichloro-3,3,3-trifluoropropane (HCFC-243 db; CF3 CHClCH₂Cl); 2-chloro-1,1,1-trifluoropropane (HCFC-253 db; CF3-CHCl—CH3), 3-chloro-1,1,1-trifluoropropane (HCFC-253fb; CF3-CH2-CH2Cl), 1,1,1,2-tetrafluoropropane (HFC-254eb; CF3-CHF—CH3), 1,1,1-trifluoropropane (HFC-263fb; CF3CH2CH3), 2-chloro-3,3,3-trifluoropropene (HCFO-1233xf; CF3-CCl═CH2), 1,2-difluoro-3,3,3-trifluoropropene (HCFO-1223xd; CF3CCl═CHCl); 2,3-dichloro-3,3-difluoropropene (HCFO-1232xf); CClF2CCl—CH2); 2,2-chloro 2-fluoropropene (CCl2FCH—CH2; HCFC-1241zf), 1-chloro-3,3,3-trifluoropropene (HCFO-1233zd; CF3-CH2═CHCl), isomers of trichloropropene (HCO-1240; C3H3Cl3), 1,1,2,2-tetrachloroethylene (PCE; CCl2=CCl2) or combinations thereof.

HFO-1243zf is known to form a binary azeotrope composition with HF, as disclosed in International Application Publication No. WO2009/105517, the disclosure of which is incorporated herein by reference in its entirety. The azeotrope composition comprises about 72.0 mole % HFO-1243zf and about 28.0 mole % HF at 29.8 °C and 106.6 psia (735 kPa). Further, the azeotrope composition comprises about 76.2 mole % HFC1243zf and about 23.8 mole % HF at 79.7 °C and 363 psia (2503 kPa).

Surprisingly, it has been found that HFC-254fb also forms a binary azeotrope with HF.

For purposes of this disclosure, “effective amount” is defined as the amount of each component of the inventive compositions which, when combined, results in the formation of an azeotrope or azeotrope-like composition. This definition includes the amounts of each component, which amounts may vary depending on the pressure applied to the composition so long as the azeotrope or azeotrope-like compositions continue to exist at the different pressures, but with possible different boiling points. Therefore, effective amount includes the amounts, such as may be expressed in weight percentages, of each component of the compositions of the instant disclosure which form azeotrope or azeotrope-like compositions at temperatures or pressures other than as described herein.

For the purposes of this disclosure, azeotrope or constant-boiling is intended to mean also essentially azeotrope or essentially-constant boiling. In other words, included within the meaning of these terms are not only the true azeotropes described above, but also other compositions containing the same components in different proportions, which are true azeotropes at other temperatures and pressures, as well as those equivalent compositions which are part of the same azeotrope system and are azeotrope-like in their properties. As is well recognized in this art, there is a range of compositions which contain the same components as the azeotrope, which will not only exhibit essentially equivalent properties for refrigeration and other applications, but which will also exhibit essentially equivalent properties to the true azeotrope composition in terms of constant boiling characteristics or tendency not to segregate or fractionate on boiling.

It is possible to characterize, in effect, a constant boiling admixture which may appear under many guises, depending upon the conditions chosen, by any of several criteria: The composition can be defined as an azeotrope of A, B, C (and D . . . ) since the very term “azeotrope” is at once both definitive and limitative, and requires that effective amounts of A, B, C (and D . . . ) for this unique composition of matter which is a constant boiling composition. It is well known by those skilled in the art, that, at different pressures, the composition of a given azeotrope will vary at least to some degree, and changes in pressure will also change, at least to some degree, the boiling point temperature. Thus, an azeotrope of A, B, C (and D . . . ) represents a unique type of relationship but with a variable composition which depends on temperature and/or pressure. Therefore, compositional ranges, rather than fixed compositions, are often used to define azeotropes. The composition can be defined as a particular weight percent relationship or mole percent relationship of A, B, C (and D . . . ), while recognizing that such specific values point out only one particular relationship and that in actuality, a series of such relationships, represented by A, B, C (and D . . . ) actually exist for a given azeotrope, varied by the influence of pressure. An azeotrope of A, B, C (and D . . . ) can be characterized by defining the compositions as an azeotrope characterized by a boiling point at a given pressure, thus giving identifying characteristics without unduly limiting the scope of the invention by a specific numerical composition, which is limited by and is only as accurate as the analytical equipment available.

When there are two azeotropes, the composition can be defined as an azeotrope of A which is HFO-1243zf/HF and B which is HFC-254fb/HF, or vice versa since the very term “azeotrope” is at once both definitive and limitative and requires that effective amounts of A and B for this unique composition of matter which is a constant boiling composition. It is well known by those skilled in the art, that, at different pressures, the composition of a given azeotrope will vary at least to some degree, and changes in pressure will also change, at least to some degree, the boiling point temperature. Thus, the azeotropes of A and B represent a unique type of relationship but with a variable composition which depends on temperature and/or pressure. Therefore, compositional ranges, rather than fixed compositions, are often used to define azeotropes. The composition can be defined as a particular weight percent relationship or mole percent relationship of A and B, while recognizing that such specific values point out only one particular relationship and that in actuality, a series of such relationships, represented by A and B actually exist for a given azeotrope, varied by the influence of pressure. An azeotrope of A and B can be characterized by defining the compositions as an azeotrope characterized by a boiling point at a given pressure, thus giving identifying characteristics without unduly limiting the scope of the invention by a specific numerical composition, which is limited by and is only as accurate as the analytical equipment available.

The azeotrope or azeotrope-like compositions of the present disclosure can be prepared by any convenient method including mixing or combining the desired amounts. A preferred method is to weigh the desired component amounts and thereafter combine them in an appropriate container. Another preferred method is to form the azeotrope or azeotrope-like compositions as the distillate stream of a distillation column.

It was unexpected that HFC-254fb would form an azeotrope or azeotrope-like composition with HF.

In some embodiments, a process is provided for separating hydrogen fluoride (HF) from a composition (e.g., a process stream) comprising 1,1,1,3-tetrafluoropropane (HFC-254fb), 3,3,3-trifluoropropene (HFO-1243zf), and a molar excess of HF. The process comprises subjecting the process stream to distillation in a first distillation column under the boiling conditions of an HFO-1243zf/HF azeotrope or near-azeotrope and an HFC-254fb/HF azeotrope or near-azeotrope. The process also comprises forming a first distillate stream comprising an azeotrope or near-azeotrope forming composition of HFC-254fb/HF and HFO-1243zf/HF and forming a first bottoms stream of HF essentially free of HFC-254fb and HFO-1243zf from the first distillation column.

In some embodiments, the process of the above paragraph further comprises condensing, cooling, and decanting the first distillate stream to form an HF-enriched stream and an HFC-254fb-rich stream and recycling the HF-enriched stream to the first distillation column.

In some embodiments, the process of the above paragraph further comprises subjecting the HFC-254fb-rich stream to distillation in a second distillation column under the boiling conditions of HFC-254fb/HF and HFO-1243zf/HF azeotropes or near-azeotropes and forming a second distillate stream comprising an azeotrope or near-azeotrope composition of HFC-254fb/HF and HFO-1243zf/HF and a second bottoms stream of HFC-254fb and HFO-1243zf essentially free of HF from the second distillation column.

In some embodiments, the process further comprises subjecting the second bottoms stream to a third distillation column to form an HFC-254fb stream essentially free of HFO-1243zf using known methods.

In some embodiments, the process stream of any of the above paragraphs is a reaction product stream of a reaction process to produce HFO-1243zf by fluorination of 1,1,1,3-tetrachloropropane (HCC-250fb) with HF.

In some embodiments, a process is provided for separating 1,1,1,3-tetrafluoropropane (HFC-254fb) from an HFC-254fb-rich stream comprising HFC-254fb, 3,3,3-trifluoropropene (HFO-1243zf), and hydrogen fluoride (HF). The process comprises subjecting an HFC-254fb-rich stream to distillation in a second distillation column under the boiling conditions of HFC-254fb/HF and HFO-1243zf/HF azeotropes or near-azeotropes which forms a second distillate overhead stream and a second bottoms stream. The second bottoms stream is then subjected to a third distillation column to form an HFC-254fb stream essentially free of HFO-1243zf.

In one embodiment, the composition disclosed is an azeotrope or near-azeotrope composition consisting essentially of 1,1,1,3-tetrafluoropropane (HFC-254fb) and hydrogen fluoride (HF).

In one embodiment, the composition disclosed is an azeotropic or near-azeotropic composition consisting essentially of about 6.0 to about 25.6 mole percent HFC-254fb and from about 94.0 to about 74.4 mole percent HF. In one embodiment, the composition further optionally includes at least one of 3-chloro-3,3-difluoro-1-propene (HCFO-1242zf) and HFO-1243zf.

In some embodiments, the composition disclosed is an azeotropic or near-azeotropic composition consisting essentially of about 6.0 to about 25.6 mole percent HFC-254fb and from about 94.0 to about 74.4 mole percent HF, and having a vapor pressure of from about 3.0 psia (20.7 kPa) to about 108 psia (744.6 kPa) at a temperature from about −20 °C to about 80 °C. In one embodiment, the composition further optionally includes at least one of HCFO-1242zf and HFO-1243zf.

In one embodiment, the composition disclosed is an azeotrope or near-azeotrope composition consisting essentially of about 18.7 mol % HFC-254fb and about 81.3 mol % HF and has a vapor pressure of about 24.5 psia (169 kPa) at a temperature of about 30 °C, and optionally includes at least one of HCFO-1242zf and HFO-1243zf.

In one embodiment, the composition disclosed herein is an azeotrope or near-azeotrope consisting essentially of: 1,1,1,3-tetrafluoropropane (HFC-254fb) and HF, and optionally at least one of HCFO-1242zf and HFO-1243zf.

In some embodiments, a process is provided for separating 1,1,1,3-tetrafluoropropane (HFC-254fb) from a process stream comprising HFC-254fb, HFO-1243zf, and a molar excess of HF. The process comprises subjecting the process stream to distillation in a first distillation column under the boiling conditions for forming HFC-254fb and HFO-1243zf azeotropes with HF, with a molar excess of HF sufficient to form both azeotropes. The process also comprises forming a first distillate stream comprising a composition of HFC-254fb, HFO-1243zf, and a sufficient amount of HF to form azeotrope or near-azeotrope compositions between HFC-254fb and HF and between HFO-1243zf and HF, and a first bottoms stream of HF essentially free of HFC-254fb and HFO-1243zf from the first distillation column.

In some embodiments, the process comprises the product stream from the production of HFO-1243zf, e.g., by fluorination of 1,1,1,3-tetrachloropropane (HCC-250fb) with HF.

In some embodiments, the process stream of any of the above paragraphs further comprises 3-chloro-3,3-difluoro-1-propene (HCFO-1242zf).

In some embodiments, the first distillate stream and the second distillation stream of any of the above paragraphs comprise an azeotropic or near-azeotropic composition comprising about 6.0 to about 25.6 mole percent HFC-254fb and from about 94.0 to about 74.4 mole percent HF.

In some embodiments, the first distillate stream and the second distillation stream of any of the above paragraphs comprise an azeotropic or near-azeotropic composition comprising about 6.0 to about 25.6 mole percent HFC-254fb and from about 94.0 to about 74.4 mole percent HF and having a vapor pressure of from about 3.0 psia (20.7 kPa) to about 108 psia (744.6 kPa) at a temperature from about −20 °C to about 80 °C.

In some embodiments, the first distillate stream and the second distillation stream of any of the above paragraphs comprise about 18.7 mol % HFC-254fb and about 81.3 mol % HF and have a vapor pressure of about 24.5 psia (169 kPa) at a temperature of about 30 °C.

In some embodiments, compositions comprising azeotropes or near-azeotropes of 1,1,1,3-tetrafluoropropane (HFC-254fb)/HF and 3,3,3-trifluoropropene (HFO-1243zf)/HF are provided.

In some embodiments, the composition comprises about 6.0 to about 25.6 mole percent HFC-254fb and from about 94.0 to about 74.4 mole percent HF.

In some embodiments, the composition comprises about 6.0 to about 25.6 mole percent HFC-254fb and from about 94.0 to about 74.4 mole percent HF and having a vapor pressure of from about 3.0 psia (20.7 kPa) to about 108 psia (744.6 kPa) at a temperature from about −20 °C to about 80 °C.

In some embodiments, the composition comprises about 18.7 mol % HFC-254fb and about 81.3 mol % HF and has a vapor pressure of about 24.5 psia (169 kPa) at a temperature of about 30 °C.

In some embodiments, the composition described herein further comprises 3-chloro-3,3-difluoro-1-propene (HCFO-1242zf).

### Examples

Referring to Example 1 illustrated in the FIGURE, the process stream 100 comprising HFC-254fb and HFO-1243zf and a molar excess of HF is fed to a first distillation column 110. This first column 110 is operated under appropriate conditions to approach the boiling conditions of HFC-254fb and HFO-1243zf azeotropes with HF. Because HF is being fed to the first column 110 in excess of that needed to form azeotropes with HFC-254fb and HFO-1243zf, excess HF, essentially free of HFC-254fb and HFO-1243zf, is recovered as the bottoms of the first column 110 as a first bottoms stream 120. The overhead stream 130, rich in HFC-254fb and HFO-1243zf, is fed to condenser 140 and withdrawn as condensed stream 150. Stream 150 is split into streams 190 and 200. Stream 190 is returned as reflux to column 110 and stream 200 is further processed, e.g., distilling in a second distillation. Details for operating the system of the FIGURE are identified in Table 2.

TABLE 2

| STREAM | 100 | 120 | 130 | 150 | 190 | 200 |
|---|---|---|---|---|---|---|
| flow (lb/hr) | 100 | 31.2 | 206.376 | 206.376 | 192.3 | 68.8 |
| temp (°C) | 51.2 | 67.2 | 37.4 | 17.8 | 17.8 | 17.8 |
| pres (psia) | 152.4 | 65.7 | 65.7 | 65.7 | 65.7 | 65.7 |
| HF wt % | 45 | 99.995 | 20.1 | 20.1 | 20.1 | 20.1 |
| 254fb wt % | 10 | 50 ppmw | 14.5 | 14.5 | 14.5 | 14.5 |
| 1243zf wt % | 45 | trace | 65.4 | 65.4 | 65.4 | 65.4 |

Distillation column 110 30 theoretical stages
Feed stream 110 enters at stage 15
Mass reflux ratio of 2
Pressure is 65.7 psia
Bottom temperature 67.2 °C

In separation processes described herein that include more than one distillation column, the distillation columns may all operate at the same pressure and/or temperature or at different pressures and/or temperatures.

Where the composition to be separated is a reaction product stream formed by fluorination of HCC-250fb with HF, it is desirable to recycle any unreacted HFC-254fb back to the reactor so that it may be converted to HFO-1243zf. However, it is necessary that HFO-1243zf be removed from the unreacted HFC-254fb prior to being recycled so as not to inhibit the equilibrium reaction. It is also necessary that the HF be removed from the HFO-1243zf to allow its use as a reagent in another reaction or as a refrigerant or in other applications.

### OTHER EMBODIMENTS

Forming a first distillate stream sufficient for forming an azeotrope or near-azeotrope composition comprising HFC-254fb and HF, and a first bottoms stream of HF containing less than 100 ppm HFC-254fb and HFO-1243zf from the first distillation column.

Forming a first distillate stream sufficient for forming an azeotrope or near-azeotrope composition comprising HFC-254fb and HF, and a first bottoms stream of HF containing less than 10 ppm HFC-254fb and HFO-1243zf from the first distillation column.

Forming a first distillate stream sufficient for forming an azeotrope or near-azeotrope composition comprising HFC-254fb and HF, and a first bottoms stream of HF containing less than 1 ppm HFC-254fb and HFO-1243zf from the first distillation column.

Forming a first distillate stream sufficient for forming an azeotrope or near-azeotrope composition comprising HFC-254fb/HF and HFO-1243zf/HF, and a first bottoms stream of HF containing less than 100 ppm HFC-254fb and HFO-1243zf from the first distillation column.

Forming a first distillate stream sufficient for forming an azeotrope or near-azeotrope composition comprising HFC-254fb/HF and HFO-1243zf/HF, and a first bottoms stream of HF containing less than 10 ppm HFC-254fb and HFO-1243zf from the first distillation column.

Forming a first distillate stream sufficient for forming an azeotrope or near-azeotrope composition comprising HFC-254fb/HF and HFO-1243zf/HF, and a first bottoms stream of HF containing less than 1 ppm HFC-254fb and HFO-1243zf from the first distillation column.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any sub combination. Further, references to values stated in ranges include each and every value within that range.

1. A process for separating hydrogen fluoride (HF) from a process stream comprising 1,1,1,3-tetrafluoropropane (HFC-254fb), 3,3,3-trifluoropropene (HFO-1243zf), and a molar excess of HF, the process comprising:
subjecting said process stream to distillation in a first distillation column under the boiling conditions of HFC-254fb and HFO-1243zf azeotropes with HF; and
forming a first distillate stream comprising azeotropes or near-azeotropes of HFC-254fb/HF and HFO-1243zf/HF and a first bottoms stream of HF essentially free of HFC-254fb and HFO-1243zf from the first distillation column.

2. The process of claim 1, further comprising converting the first distillate stream into an HF-enriched stream and an HFC-254fb-rich stream.

3. The process of claim 2, wherein said converting comprises cooling said first distillate stream to form said HF-enriched stream and said HFC-254fb-rich stream.

4. The process of claim 3, further comprising feeding said HF-enriched stream to said first distillation column.

5. The process of claim 2, wherein said converting comprises at least one of condensing and cooling the first distillate stream to form said HF-enriched stream and said HFC-254fb-rich stream.

6. The process of claim 1, wherein said process stream is a reaction product stream of a reaction process to produce HFO-1243zf by fluorination of 1,1,1,3-tetrachloropropane (HCC-250fb) with HF.

7. The process of claim 1, wherein said process stream further comprises 3-chloro-3,3-difluoro-1-propene (HCFO-1242zf).

8. The process of claim 1, wherein said first distillate stream and said second distillate stream comprise about 6.0 to about 25.6 mol % HFC-254fb and from about 94.0 to about 74.4 mol % HF.

9. The process of claim 1, wherein said first distillate stream and said second distillate stream comprise about 6.0 to about 25.6 mol % HFC-254fb and from about 94.0 to about 74.4 mol % HF and have a vapor pressure of from about 3.0 psia (20.7 kPa) to about 108 psia (744.6 kPa) at a temperature from about −20 °C to about 80 °C.

10. The process of claim 1, wherein said first distillate stream and said second distillate stream comprise about 18.7 mol % HFC-254fb and about 81.3 mol % HF and have a vapor pressure of about 24.5 psia (169 kPa) at a temperature of about 30 °C.

11. A process for separating 1,1,1,3-tetrafluoropropane (HFC-254fb) from a stream comprising HFC-254fb, 3,3,3-trifluoropropene (HFO-1243zf), and hydrogen fluoride (HF), the process comprising:
subjecting said stream to distillation in a first distillation column under the boiling conditions of HFC-254fb/HF and HFO-1243zf/HF azeotropes;
forming a first distillate stream comprising azeotropic or near-azeotropic compositions of HFC-254fb/HF and HFO-1243zf/HF and a first bottoms stream of HFC-254fb and HFO-1243zf essentially free of HF from the first distillation column; and
subjecting the first bottoms stream to a second distillation column to form an HFC-254fb stream essentially free of HFO-1243zf.

12. The process of claim 11, wherein said stream is an HFC-254fb rich stream.

13. The process of claim 12, wherein said HFC-254fb-rich stream further comprises 3-chloro-3,3-difluoro-1-propene (HCFO-1242zf).

14. The process of claim 11, wherein said first distillate stream comprises about 6.0 to about 25.6 mol % HFC-254fb and from about 94.0 to about 74.4 mol % HF.

15. The process of claim 11, wherein said first distillate stream comprises about 6.0 to about 25.6 mol % HFC-254fb and from about 94.0 to about 74.4 mol % HF and has a vapor pressure of from about 3.0 psia (20.7 kPa) to about 108 psia (744.6 kPa) at a temperature from about −20 °C to about 80 °C.

16. The process of claim 11, wherein said first distillate stream comprises about 18.7 mol % HFC-254fb and about 81.3 mol % HF and have a vapor pressure of about 24.5 psia (169 kPa) at a temperature of about 30 °C.

17. An azeotrope or near-azeotrope composition comprising 1,1,1,3-tetrafluoropropane (HFC-254fb) and hydrogen fluoride (HF).

18. The composition of claim 17, wherein said composition comprises about 6.0 to about 25.6 mol % HFC-254fb and from about 94.0 to about 74.4 mol % HF.

19. The composition of claim 17, wherein said composition about 6.0 to about 25.6 mol % HFC-254fb and from about 94.0 to about 74.4 mol % HF, and has a vapor pressure of from about 3.0 psia (20.7 kPa) to about 108 psia (744.6 kPa) at a temperature from about −20 °C to about 80 °C.

20. The composition of claim 17, wherein said composition comprises about 18.7 mol % HFC-254fb and about 81.3 mol % HF and have a vapor pressure of about 24.5 psia (169 kPa) at a temperature of about 30 °C.

21. (canceled)

22. (canceled)

23. A composition comprising: i) the azeotrope or near-azeotrope composition of claim 17, ii) 1242zf; and iii) 1243zf.

24. A separation process comprising:
passing a process stream comprising 1,1,1,3-tetrafluoropropane (HFC-254fb), 3,3,3-trifluoropropene (HFO-1243zf), and HF through a distillation column;
controlling the HF content of the process stream to form azeotrope and near-azeotrope HFC-254fb/HF compositions; and
separating at least HFC-254fb.

25. The separation process according to claim 24, wherein the azeotrope and near-azeotrope HFC-254fb/HF compositions are used to separate HFC-254fb.

26. The separation process of claim 24, the process further comprising recovering an HF stream essentially free of HFC-254fb.

27. The process of claim 24, the process further comprising recovering an HF stream essentially free of at least one of HFO-1243zf and HFC-254fb.

28. The process according to claim 1, wherein essentially free is one of: less than about 100 ppm (mole basis), less than about 10 ppm, or less than about 1 ppm of at least one of HFO-1243zf and HFC-254fb.

29. An azeotrope or near-azeotrope composition consisting essentially of 1,1,1,3-tetrafluoropropane (HFC-254fb) and hydrogen fluoride (HF).

30. The composition of claim 29, wherein said composition consists essentially of about 6.0 to about 25.6 mol % HFC-254fb and from about 94.0 to about 74.4 mol % HF.

31. The composition of claim 29, wherein said composition consists essentially of about 6.0 to about 25.6 mol % HFC-254fb and from about 94.0 to about 74.4 mol % HF and has a vapor pressure of from about 3.0 psia (20.7 kPa) to about 108 psia (744.6 kPa) at a temperature from about −20 °C to about 80 °C.

32. The composition of claim 29, wherein said composition consists essentially of about 18.7 mol % HFC-254fb and about 81.3 mol % HF and have a vapor pressure of about 24.5 psia (169 kPa) at a temperature of about 30 °C.

33. A composition comprising the composition of claim 29 and 3-chloro-3,3-difluoro-1-propene (HCFO-1242zf)

34. A composition comprising the composition of claim 20 and HCFO-1243zf.

35. A composition consisting essentially of i) the azeotrope or near-azeotrope composition of claim 17, ii) 1242zf and; iii) 1243zf.

36. The process according to claim 11 wherein essentially free is one of: less than about 100 ppm (mole basis), less than about 10 ppm, or less than about 1 ppm of at least one of HF and HFO-1243zf.

37. The process according to claim 26 wherein essentially free is one of: less than about 100 ppm (mole basis), less than about 10 ppm, or less than about 1 ppm of HFC-254fb.

38. The process according to claim 27 wherein essentially free is one of: less than about 100 ppm (mole basis), less than about 10 ppm, or less than about 1 ppm of at least one of HFO-1243zf and HFC-254fb.
